(19) United States
(12) Patent Application Publication
(10) Pub. No.: US 2021/0115389 A1
(43) Pub. Date: Apr. 22, 2021

(54) SEPARATION OF ACETATE FROM FERMENTATION BROTH

(71) Applicant: LanzaTech, Inc., Skokie, IL (US)

(72) Inventor: Richard R. Rosin, Skokie, IL (US)

(21) Appl. No.: 17/074,342

(22) Filed: Oct. 19, 2020

Related U.S. Application Data
(60) Provisional application No. 62/924,666, filed on Oct. 22, 2019.

(51) Int. Cl. C12M 1/00; B01D 15/18; B01D 15/36; B01D 53/84; C12P 7/56; C12P 7/54; C07C 67/04; C08F 218/08; C08F 216/06

(52) U.S. Cl. CPC C12M 47/10; B01D 15/1821; B01D 15/363; B01D 53/84; B01D 2251/95; C12P 7/54; C07C 67/04; C08F 218/08; C08F 216/06; C12P 7/56

(57) ABSTRACT

The method of the disclosure comprises fermenting a gas substrate and a microorganism to generate a fermentation broth comprising the microorganism and the target component; passing the fermentation broth to a separation unit having an ion exchange resin in a continuous ion exchange simulated moving bed; selectively retaining the target component through ion exchange with the resin while passing the microorganism through the bed; regenerating the ion exchange resin; and recovering the target component. Alternatively, the fermentation broth is passed to a first separation zone to separate and recycle a first portion of the fermentation broth comprising the microorganism to the bioreactor and then a second portion of the fermentation broth is passed to a second separation zone comprising ion exchange resin which selectively retains the target component through ion exchange with the resin. The remainder is passed through. The ion exchange resin is regenerated, and the target component recovered.

### CROSS REFERENCE

This application claims the benefit of U.S. Provisional Application Ser. No. 62/924,666 filed Oct. 22, 2019. The content of the provisional application is expressly incorporated herein by reference in their entirety.

### FIELD

This application relates to the separation of target component(s) from a fermentation broth resulting from the culturing of a microorganism in the presence of a gas substrate, the separation using continuous ion exchange operated in simulated moving bed mode.

### BACKGROUND

Mitigation of impending climate change requires drastic reductions in emissions of greenhouse gases (GHGs), such as those generated through the burning of fossil fuels like coal and oil. Although sustainable sources of chemicals and transportation fuels are currently insufficient to significantly displace our dependence on fossil carbon, gas fermentation has recently emerged as an alternative platform for the biological fixation of such gases such as carbon dioxide (CO₂), carbon monoxide (CO), and/or hydrogen (H₂) into sustainable fuels and chemicals. In particular, gas fermentation technology can utilize a wide range of feedstocks including gasified organic matter (e.g., municipal solid waste or agricultural waste) or industrial waste gases (e.g., from steel mills or oil refineries) to produce ethanol, jet fuel, and a variety of other products. Gas fermentation alone could displace 30% of crude oil use and reduce global CO₂ emissions by 10%, but, as with any disruptive technology, many technical challenges must be overcome before this potential is fully achieved.

### SUMMARY

A method for separating a target component from a fermentation broth is disclosed. The method comprises fermenting a gas substrate and a microorganism to generate a fermentation broth comprising the microorganism and the target component; passing the fermentation broth to a separation unit having an ion exchange resin in a continuous ion exchange simulated moving bed; selectively retaining the target component through ion exchange with the resin and passing the microorganism through the continuous ion exchange simulated moving bed; regenerating the ion exchange resin; and recovering the target component. The target component may be the conjugate base of any low molecular weight organic acid such as formate, acetate, lactate, or both. The target component may be reacted to form one or more products. The microorganism may be derived from a parental microorganism selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui. The microorganism may be a member of the genus Clostridium. The microorganism may be derived from Clostridium autoethanogenum, Clostridium ljungdahlii, Clostridium ragsdalei, or Clostridium coskatii. The gas substrate may be industrial waste gas, industrial off gas, syngas, gasified waste, or gasified biomass. The target compound may be acetate and the one or more products may be vinyl acetate. The vinyl acetate may be further reactor to form polyvinyl acetate or polyvinyl alcohol.

A composition comprising a component derived from the vinyl acetate reacted from the acetate recovered by the method is disclosed. The composition may be a polymer, a copolymer, an adhesive, a coating, a paint, a film, a textile, a foam, a wire insulation or a cable insulation.

A further method for separating a target component from a fermentation broth is disclosed. The method comprises fermenting a gas substrate and a microorganism to generate a fermentation broth comprising the microorganism and the target component; passing the fermentation broth to a first separation zone to separate and recycle a first portion of the fermentation broth comprising the microorganism to the bioreactor; passing a second portion of the fermentation broth to a second separation zone comprising ion exchange resin; selectively retaining the target component through ion exchange with the resin and passing remainder through the second separation zone; and regenerating the ion exchange resin with a regenerate and recovering the target component. The regenerate may comprise at least a portion of the remainder or may be derived from the remainder. The target component may be a conjugate base of a low molecular weight organic acid. The target component may be acetate, lactate, or both. The ion exchange resin may be a strong anion exchange resin. The gas substrate may be industrial waste gas, industrial off gas, synthesis gas derived from gasified waste, synthesis gas derived from gasified biomass, or any combination thereof. The target component may be reacted to form one or more products. The target compound may be acetate and the one or more products may be vinyl acetate. The vinyl acetate may be reacted to form polyvinyl acetate or polyvinyl alcohol. The polyvinyl acetate or polyvinyl alcohol may be used to form a polymer, a copolymer, an adhesive, a coating, a paint, a film, a textile, a foam, a wire insulation or a cable insulation.

A biological conversion apparatus is disclosed, the biological conversion apparatus comprising: a bioreactor system comprising an inlet to a bioreactor for containing a culture medium and microorganism to metabolize a carbon source in the substrate and produce a product and an outlet from the bioreactor; a separation zone comprising a first inlet in fluid communication with the outlet of the bioreactor, an expanded bed of ion exchange resin in a simulated moving bed configuration, a second inlet in fluid communication with a regenerate source, an outlet in fluid communication with the bioreactor system, and a product outlet.

A further biological conversion apparatus is disclosed. The apparatus comprises a bioreactor system comprising an inlet to a bioreactor containing a culture medium and microorganisms to metabolize a carbon source in a substrate and produce a product and an outlet from the bioreactor; a first separation zone comprising an inlet in fluid communication with the outlet of the bioreactor, a membrane for the separation of microbial biomass, a retentate outlet in fluid communication with the bioreactor, and a permeate outlet; and a second separation zone comprising a first inlet in fluid communication with the permeate outlet of the first separation zone, at least one bed of ion exchange resin, a second inlet in fluid communication with a regenerate source, an outlet in fluid communication with the regenerate source, and a product outlet.

### BRIEF DESCRIPTION OF THE DRAWING

FIG. 1 represents a biological conversion apparatus having bioreactor system and a separation zone according to one embodiment of the disclosure.

FIG. 2 represents a biological conversion apparatus having bioreactor system, a first separation zone, and a second separation zone according to one embodiment of the disclosure.

### DESCRIPTION

The disclosure addresses the problem of separating fermentation products from the fermentation broth. Particularly the disclosure is directed to separating low molecular weight acids and/or their conjugate base that may be present in the fermentation broth. The low molecular weight acids may be separated from the fermentation broth using techniques such as ion exchange, distillation, esterification followed by distillation, or liquid-liquid extraction. Another suitable technique is referred to as “salting out” which is a purification method that utilizes the reduced solubility of certain molecules in a solution of very high ionic strength. Although, details of the disclosure are explained in reference to the ion exchange technique, any of the above separation techniques may be employed to separate low molecular weight acids and/or their conjugate base.

One exemplary product, also referred to as a target component, to be separated from the fermentation broth is acetate which partially dissociates in water from acetic acid. Another exemplary product, also referred to as a target component, to be separated from the fermentation broth is lactate which partially dissociates in water from lactic acid. One exemplary product to be separated from the fermentation broth is formate which partially dissociates in water from formic acid. Acetate, recovered from fermentation broth, may be readily converted to acetic acid which is a primary reactant in the formation of vinyl acetate which is also referred to as vinyl acetate monomer (VAM). VAM is an important commercial product as VAM may be polymerized to form polyvinyl acetate. Thus, VAM is important in the industrial production of polymers and resins that are used to produce adhesives, coatings, paints, films, textiles, foam, wire insulation and cable insulation. Acetic acid may also be used in food products and in reactions in the silica chemistry field. Similarly, lactate, recovered from fermentation broth, may be readily converted to lactic acid which is an ingredient in may skin care products. Lactic acid is added to skin care products to enhance the skin lightening effects, improve collegan and elastin synthesis, and accelerate exfoliation cell renewal. Rising demand for anti-acne and anti-aging products is expected to spur product demand for lactic acid.

Unless otherwise defined, the following terms as used throughout this specification are defined as follows:

The terms “increasing the efficiency,” “increased efficiency,” and the like, when used in relation to a fermentation process, include, but are not limited to, increasing one or more of the rate of growth of microorganisms catalyzing the fermentation, the growth and/or product production rate at elevated product concentrations, increasing the volume of desired product produced per volume of substrate consumed, increasing the rate of production or level of production of the desired product, increasing the relative proportion of the desired product produced compared with other by-products of the fermentation, decreasing the amount of water consumed by the process, and decreasing the amount of energy utilized by the process.

The term “fermentation” should be interpreted as a metabolic process that produces chemical changes in a substrate. For example, a fermentation process receives one or more substrates and produces one or more products through utilization of one or more microorganisms. The term “fermentation,” “gas fermentation” and the like should be interpreted as the process which receives one or more substrate, such as syngas produced by gasification and produces one or more product through the utilization of one or more C1-fixing microorganism. Preferably the fermentation process includes the use of one or more bioreactor. The fermentation process may be described as either “batch” or “continuous”. “Batch fermentation” is used to describe a fermentation process where the bioreactor is filled with raw material, e.g. the carbon source, along with microorganisms, where the products remain in the bioreactor until fermentation is completed. In a “batch” process, after fermentation is completed, the products are extracted, and the bioreactor is cleaned before the next “batch” is started. “Continuous fermentation” is used to describe a fermentation process where the fermentation process is extended for longer periods of time, and product and/or metabolite is extracted during fermentation. Preferably the fermentation process is continuous.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

Metabolic engineering of microorganisms, such as Clostridia, can tremendously expand their ability to produce many important fuel and chemical molecules other than native metabolites, such as ethanol. However, until recently, Clostridia were considered genetically intractable and therefore generally off limits to extensive metabolic engineering efforts. In recent years several different methods for genome engineering for Clostridia have been developed including intron-based methods (ClosTron) (Kuehne, Strain Eng: Methods and Protocols, 389-407, 2011), allelic exchange methods (ACE) (Heap, Nucl Acids Res, 40: e59, 2012; Ng, PLoS One, 8: e56051, 2013), Triple Cross (Liew, Frontiers Microbiol, 7: 694, 2016), methods mediated through I-SceI (Zhang, Journal Microbiol Methods, 108: 49-60, 2015), MazF (Al-Hinai, Appl Environ Microbiol, 78: 8112-8121, 2012), or others (Argyros, Appl Environ Microbiol, 77: 8288-8294, 2011), Cre-Lox (Ueki, mBio, 5: e01636-01614, 2014), and CRISPR/Cas9 (Nagaraju, Biotechnol Biofuels, 9: 219, 2016). However, it remains extremely challenging to iteratively introduce more than a few genetic changes, due to slow and laborious cycling times and limitations on the transferability of these genetic techniques across species. Furthermore, we do not yet sufficiently understand C1 metabolism in Clostridia to reliably predict modifications that will maximize C1 uptake, conversion, and carbon/energy/redox flows towards product synthesis. Accordingly, introduction of target pathways in Clostridia remains a tedious and time-consuming process.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms.

“Wild type” refers to the typical form of an organism, strain, gene, or characteristic as it occurs in nature, as distinguished from mutant or variant forms.

“Endogenous” refers to a nucleic acid or protein that is present or expressed in the wild-type or parental microorganism from which the microorganism of the disclosure is derived. For example, an endogenous gene is a gene that is natively present in the wild-type or parental microorganism from which the microorganism of the disclosure is derived. In one embodiment, the expression of an endogenous gene may be controlled by an exogenous regulatory element, such as an exogenous promoter.

“Exogenous” refers to a nucleic acid or protein that originates outside the microorganism of the disclosure. For example, an exogenous gene or enzyme may be artificially or recombinantly created and introduced to or expressed in the microorganism of the disclosure. An exogenous gene or enzyme may also be isolated from a heterologous microorganism and introduced to or expressed in the microorganism of the disclosure. Exogenous nucleic acids may be adapted to integrate into the genome of the microorganism of the disclosure or to remain in an extra-chromosomal state in the microorganism of the disclosure, for example, in a plasmid.

“Heterologous” refers to a nucleic acid or protein that is not present in the wild-type or parental microorganism from which the microorganism of the disclosure is derived. For example, a heterologous gene or enzyme may be derived from a different strain or species and introduced to or expressed in the microorganism of the disclosure. The heterologous gene or enzyme may be introduced to or expressed in the microorganism of the disclosure in the form in which it occurs in the different strain or species. Alternatively, the heterologous gene or enzyme may be modified in some way, e.g., by codon-optimizing it for expression in the microorganism of the disclosure or by engineering it to alter function, such as to reverse the direction of enzyme activity or to alter substrate specificity.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “polypeptide”, “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Mutated” refers to a nucleic acid or protein that has been modified in the microorganism of the disclosure compared to the wild-type or parental microorganism from which the microorganism of the disclosure is derived. In one embodiment, the mutation may be a deletion, insertion, or substitution in a gene encoding an enzyme. In another embodiment, the mutation may be a deletion, insertion, or substitution of one or more amino acids in an enzyme.

In particular, a “disruptive mutation” is a mutation that reduces or eliminates (i.e., “disrupts”) the expression or activity of a gene or enzyme. The disruptive mutation may partially inactivate, fully inactivate, or delete the gene or enzyme. The disruptive mutation may be any mutation that reduces, prevents, or blocks the biosynthesis of a product produced by an enzyme. The disruptive mutation may be a knockout (KO) mutation. The disruption may also be a knockdown (KD) mutation that reduces, but does not entirely eliminate, the expression or activity of a gene, protein, or enzyme. While KOs are generally effective in increasing product yields, they sometimes come with the penalty of growth defects or genetic instabilities that outweigh the benefits, particularly for non-growth coupled products. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

Introduction of a disruptive mutation results in a microorganism of the disclosure that produces no target product or substantially no target product or a reduced amount of target product compared to the parental microorganism from which the microorganism of the disclosure is derived. For example, the microorganism of the disclosure may produce no target product or at least about 1%, 3%, 5%, 10%, 20%, 30%, 40%, 50%, 60%, 70%, 80%, 90%, or 95% less target product than the parental microorganism. For example, the microorganism of the disclosure may produce less than about 0.001, 0.01, 0.10, 0.30, 0.50, or 1.0 g/L target product.

“Codon optimization” refers to the mutation of a nucleic acid, such as a gene, for optimized or improved translation of the nucleic acid in a particular strain or species. Codon optimization may result in faster translation rates or higher translation accuracy. In a one embodiment, the genes of the disclosure are codon optimized for expression in Clostridium, particularly Clostridium autoethanogenum, Clostridium ljungdahlii, or Clostridium ragsdalei. In another embodiment, the genes of the disclosure are codon optimized for expression in Clostridium autoethanogenum LZ1561, which is deposited under DSMZ accession number DSM23693.

“Overexpressed” refers to an increase in expression of a nucleic acid or protein in the microorganism of the disclosure compared to the wild-type or parental microorganism from which the microorganism of the disclosure is derived. Overexpression may be achieved by any means known in the art, including modifying gene copy number, gene transcription rate, gene translation rate, or enzyme degradation rate.

The term “variants” includes nucleic acids and proteins whose sequence varies from the sequence of a reference nucleic acid and protein, such as a sequence of a reference nucleic acid and protein disclosed in the prior art or exemplified herein. The disclosure may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. These include homologous genes in species such as Clostridium acetobutylicum, Clostridium beijerinckii, or Clostridium ljungdahlii, the details of which are publicly available on websites such as Genbank or NCBI. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are well known in the art (e.g., Tijssen, Laboratory techniques in biochemistry and molecular biology-hybridization with nucleic acid probes, Second Chapter “Overview of principles of hybridization and the strategy of nucleic acid probe assay,” Elsevier, N.Y, 1993).

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Nucleic acids may be delivered to a microorganism of the disclosure using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In one embodiment, nucleic acids are delivered to the microorganism of the disclosure using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. Ideally, the promoter is a Wood-Ljungdahl pathway promoter, a ferredoxin promoter, a pyruvate:ferredoxin oxidoreductase promoter, an Rnf complex operon promoter, an ATP synthase operon promoter, or a phosphotransacetylase/acetate kinase operon promoter.

A “microorganism” is a microscopic organism, especially a bacterium, archaeon, virus, or fungus. The microorganism of the disclosure is typically a bacterium. As used herein, recitation of “microorganism” should be taken to encompass “bacterium.”

A “parental microorganism” is a microorganism used to generate a microorganism of the disclosure. The parental microorganism may be a naturally-occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. In one embodiment, the parental microorganism is Clostridium autoethanogenum LZ1561, which was deposited with Deutsche Sammlung von Mikroorganismen and Zellkulturen GmbH (DSMZ) located at Inhoffenstraß 7B, D-38124 Braunschwieg, Germany on Jun. 7, 2010 under the terms of the Budapest Treaty and accorded accession number DSM23693. This strain is described in International Patent Application No. PCT/NZ2011/000144, which published as WO 2012/015317.

The term “derived from” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes. Generally, the microorganism of the disclosure is derived from a parental microorganism. In one embodiment, the microorganism of the disclosure is derived from Clostridium autoethanogenum, Clostridium ljungdahlii, or Clostridium ragsdalei. In one embodiment, the microorganism of the disclosure is derived from Clostridium autoethanogenum LZ1561, which is deposited under DSMZ accession number DSM23693.

The microorganism of the disclosure may be further classified based on functional characteristics. For example, the microorganism of the disclosure may be or may be derived from a C1-fixing microorganism, an anaerobe, an acetogen, an ethanologen, a carboxydotroph, and/or a methanotroph. Table 1 provides a representative list of microorganisms and identifies their functional characteristics.

TABLE 1

| | Wood- | C1- | | | | | |
|---|---|---|---|---|---|---|---|
| | Ljungdahl | fixing | Anaerobe | Acetogen | Ethanologen | Autotroph | Carboxydotroph |
| Acetobacterium woodii | + | + | + | + | +/−¹ | + | − |
| Alkalibaculum bacchii | + | + | + | + | + | + | + |
| Blautia producta | + | + | + | + | − | + | + |
| Butyribacterium | + | + | + | + | + | + | + |
| methyl otrophicum | | | | | | | |
| Clostridium aceticum | + | + | + | + | − | + | + |
| Clostridium | + | + | + | + | + | + | + |
| autoethanogenum | | | | | | | |
| Clostridium | + | + | + | + | + | + | + |
| carboxidivorans | | | | | | | |
| Clostridium coskatii | + | + | + | + | + | + | + |
| Clostridium drakei | + | + | + | + | − | + | + |
| Clostridium | + | + | + | + | − | + | + |
| formicoaceticum | | | | | | | |
| Clostridium ljungdahlii | + | + | + | + | + | + | + |
| Clostridium magnum | + | + | + | + | − | + | +/−² |
| Clostridium ragsdalei | + | + | + | + | + | + | + |
| Clostridium scatologenes | + | + | + | + | − | + | + |
| Eubacterium limosum | + | + | + | + | − | + | + |
| Moorella | + | + | + | + | + | + | + |
| thermautotrophica | | | | | | | |
| Moorella thermoacetica | | | | | | | |
| (formerly Clostridium | | | | | | | |
| thermoaceticum) | + | + | + | + | −³ | + | + |
| Oxobacter pfennigii | + | + | + | + | − | + | + |
| Sporomusa ovata | + | + | + | + | − | + | +/−⁴ |
| Sporomusa silvacetica | + | + | + | + | − | + | +/−⁵ |
| Sporomusa sphaeroides | + | + | + | + | − | + | +/−⁶ |
| Thermoanaerobacter kivui | + | + | + | + | − | + | − |

¹Acetobacterium woodii can produce ethanol from fructose, but not from gas.
²It has not been investigated whether Clostridium magnum can grow on CO.
³One strain of Moorella thermoacetica, Moorella sp. HUC22-1, has been reported produce ethanol from gas.
⁴It has not been investigated whether Sporomusa ovata can grow on CO.
⁵It has not been investigated whether Sporomusa silvacetica can grow on CO.
⁶It has not been investigated whether Sporomusa sphaeroides can grow on CO.

“Wood-Ljungdahl” refers to the Wood-Ljungdahl pathway of carbon fixation as described, e.g., by Ragsdale, Biochim Biophys Acta, 1784: 1873-1898, 2008. “Wood-Ljungdahl microorganisms” refers, predictably, to microorganisms containing the Wood-Ljungdahl pathway. Generally, the microorganism of the disclosure contains a native Wood-Ljungdahl pathway. Herein, a Wood-Ljungdahl pathway may be a native, unmodified Wood-Ljungdahl pathway or it may be a Wood-Ljungdahl pathway with some degree of genetic modification (e.g., overexpression, heterologous expression, knockout, etc.) so long as it still functions to convert CO, CO₂, and/or H₂ to acetyl-CoA.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1 carbon source. Typically, the microorganism of the disclosure is a C1-fixing bacterium. In one embodiment, the microorganism of the disclosure is derived from a C1-fixing microorganism identified in Table 1.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen). Typically, the microorganism of the disclosure is an anaerobe. In one embodiment, the microorganism of the disclosure is derived from an anaerobe identified in Table 1.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3rd edition, p. 354, New York, N.Y., 2006). All naturally occurring acetogens are C1-fixing, anaerobic, autotrophic, and non-methanotrophic. Typically, the microorganism of the disclosure is an acetogen. In one embodiment, the microorganism of the disclosure is derived from an acetogen identified in Table 1.

An “ethanologen” is a microorganism that produces or is capable of producing ethanol. Typically, the microorganism of the disclosure is an ethanologen. In one embodiment, the microorganism of the disclosure is derived from an ethanologen identified in Table 1.

An “autotroph” is a microorganism capable of growing in the absence of organic carbon. Instead, autotrophs use inorganic carbon sources, such as CO and/or CO₂. Typically, the microorganism of the disclosure is an autotroph. In one embodiment, the microorganism of the disclosure is derived from an autotroph identified in Table 1.

A “carboxydotroph” is a microorganism capable of utilizing CO as a sole source of carbon and energy. Typically, the microorganism of the disclosure is a carboxydotroph. In one embodiment, the microorganism of the disclosure is derived from a carboxydotroph identified in Table 1.

A “methanotroph” is a microorganism capable of utilizing methane as a sole source of carbon and energy. In certain embodiments, the microorganism of the disclosure is a methanotroph or is derived from a methanotroph. In other embodiments, the microorganism of the disclosure is not a methanotroph or is not derived from a methanotroph.

More broadly, the microorganism of the disclosure may be derived from any genus or species identified in Table 1. For example, the microorganism may be a member of a genus selected from the group consisting of Acetobacterium, Alkalibaculum, Blautia, Butyribacterium, Clostridium, Eubacterium, Moorella, Oxobacter, Sporomusa, and Thermoanaerobacter. In particular, the microorganism may be derived from a parental bacterium selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui.

In one embodiment, the microorganism of the disclosure is derived from the cluster of Clostridia comprising the species Clostridium autoethanogenum, Clostridium ljungdahlii, and Clostridium ragsdalei. These species were first reported and characterized by Abrini, Arch Microbiol, 161: 345-351, 1994 (Clostridium autoethanogenum), Tanner, Int J System Bacteriol, 43: 232-236, 1993 (Clostridium ljungdahlii), and Huhnke, WO 2008/028055 (Clostridium ragsdalei).

These three species have many similarities. In particular, these species are all C1 fixing, anaerobic, acetogenic, ethanologenic, and carboxydotrophic members of the genus Clostridium. These species have similar genotypes and phenotypes and modes of energy conservation and fermentative metabolism. Moreover, these species are clustered in clostridial rRNA homology group I with 16S rRNA DNA that is more than 99% identical, have a DNA G+C content of about 22-30 mol %, are gram-positive, have similar morphology and size (logarithmic growing cells between 0.5-0.7×3-5 μm), are mesophilic (grow optimally at 30-37° C.), have similar pH ranges of about 4-7.5 (with an optimal pH of about 5.5-6), lack cytochromes, and conserve energy via an Rnf complex. Also, reduction of carboxylic acids into their corresponding alcohols has been shown in these species (Perez, Biotechnol Bioeng, 110:1066-1077, 2012). Importantly, these species also all show strong autotrophic growth on CO-containing gases, produce ethanol and acetate (or acetic acid) as main fermentation products, and produce small amounts of 2,3-butanediol and lactic acid under certain conditions.

However, these three species also have a number of differences. These species were isolated from different sources: Clostridium autoethanogenum from rabbit gut, Clostridium ljungdahlii from chicken yard waste, and Clostridium ragsdalei from freshwater sediment. These species differ in utilization of various sugars (e.g., rhamnose, arabinose), acids (e.g., gluconate, citrate), amino acids (e.g., arginine, histidine), and other substrates (e.g., betaine, butanol). Moreover, these species differ in auxotrophy to certain vitamins (e.g., thiamine, biotin). These species have differences in nucleic and amino acid sequences of Wood-Ljungdahl pathway genes and proteins, although the general organization and number of these genes and proteins has been found to be the same in all species (Köpke, Curr Opin Biotechnol, 22: 320-325, 2011).

Thus, in summary, many of the characteristics of Clostridium autoethanogenum, Clostridium ljungdahlii, or Clostridium ragsdalei are not specific to that species, but are rather general characteristics for this cluster of C1 fixing, anaerobic, acetogenic, ethanologenic, and carboxydotrophic members of the genus Clostridium. However, since these species are, in fact, distinct, the genetic modification or manipulation of one of these species may not have an identical effect in another of these species. For instance, differences in growth, performance, or product production may be observed.

The microorganism of the disclosure may also be derived from an isolate or mutant of Clostridium autoethanogenum, Clostridium ljungdahlii, or Clostridium ragsdalei. Isolates and mutants of Clostridium autoethanogenum include JA1-1 (DSM10061) (Abrini, Arch Microbiol, 161: 345-351, 1994), LZ1560 (DSM19630) (WO 2009/064200), and LZ1561 (DSM23693) (WO 2012/015317). Isolates and mutants of Clostridium ljungdahlii include ATCC 49587 (Tanner, Int J Syst Bacteriol, 43: 232-236, 1993), PETCT (DSM13528, ATCC 55383), ERI-2 (ATCC 55380) (U.S. Pat. No. 5,593,886), C-01 (ATCC 55988) (U.S. Pat. No. 6,368,819), 0-52 (ATCC 55989) (U.S. Pat. No. 6,368,819), and OTA-1 (Tirado-Acevedo, Production of bioethanol from synthesis gas using Clostridium ljungdahlii, PhD thesis, North Carolina State University, 2010). Isolates and mutants of Clostridium ragsdalei include PI 1 (ATCC BAA-622, ATCC PTA-7826) (WO 2008/028055).

“Substrate” refers to a carbon and/or energy source for the microorganism of the disclosure. Typically, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons.

The substrate generally comprises at least some amount of CO, such as about 1, 2, 5, 10, 20, 30, 40, 50, 60, 70, 80, 90, or 100 mol % CO. The substrate may comprise a range of CO, such as about 20-80, 30-70, or 40-60 mol % CO. Preferably, the substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the substrate may comprise a relatively low amount of CO, such as about 1-10 or 1-20 mol % CO. The microorganism of the disclosure typically converts at least a portion of the CO in the substrate to a product. In some embodiments, the substrate comprises no or substantially no (<1 mol %) CO.

The substrate may comprise some amount of H₂. For example, the substrate may comprise about 1, 2, 5, 10, 15, 20, or 30 mol % H₂. In some embodiments, the substrate may comprise a relatively high amount of H₂, such as about 60, 70, 80, or 90 mol % H₂. In further embodiments, the substrate comprises no or substantially no (<1 mol %) H₂.

The substrate may comprise some amount of CO₂. For example, the substrate may comprise about 1-80 or 1-30 mol % CO₂. In some embodiments, the substrate may comprise less than about 20, 15, 10, or 5 mol % CO₂. In another embodiment, the substrate comprises no or substantially no (<1 mol %) CO₂.

Although the substrate is typically gaseous, the substrate may also be provided in alternative forms. For example, the substrate may be dissolved in a liquid saturated with a CO-containing gas using a microbubble dispersion generator. By way of further example, the substrate may be adsorbed onto a solid support.

The substrate and/or C1-carbon source may be a waste gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the substrate and/or C1-carbon source may be captured from the industrial process before it is emitted into the atmosphere, using any convenient method.

The substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

In particular embodiments, the presence of hydrogen results in an improved overall efficiency of the fermentation process.

Syngas composition can be improved to provide a desired or optimum H₂:CO:CO₂ ratio. The syngas composition may be improved by adjusting the feedstock being fed to the gasification process. The desired H₂:CO:CO₂ ratio is dependent on the desired fermentation product of the fermentation process. For ethanol, the optimum H₂:CO:CO₂ ratio would be:

$$( x ) \text{:} ( y ) \text{:} ( \frac{x - 2 y}{3} ) ,$$

where x>2y, in order to satisfy the stoichiometry for ethanol production

$$( x ) {H}_{2} + ( y ) CO + ( \frac{x - 2 y}{3} ) {CO}_{2} \to ( \frac{x + y}{6} ) {C}_{2} {H}_{5} OH + ( \frac{x - y}{2} ) {H}_{2} O .$$

Operating the fermentation process in the presence of hydrogen has the added benefit of reducing the amount of CO₂ produced by the fermentation process. For example, a gaseous substrate comprising minimal H₂ will typically produce ethanol and CO₂ by the following stoichiometry [6 CO+3H₂O→C₂H₅OH+4 CO₂]. As the amount of hydrogen utilized by the C1-fixing bacterium increases, the amount of CO₂ produced decreases [e.g., 2 CO+4H₂→C₂H₅OH+H₂O].

When CO is the sole carbon and energy source for ethanol production, a portion of the carbon is lost to CO₂ as follows:

6CO+3H₂O→C₂H₅OH+4CO₂(ΔG°=−224.90 kJ/mol ethanol)

As the amount of H₂ available in the substrate increases, the amount of CO₂ produced decreases. At a stoichiometric ratio of 2:1 (H₂:CO), CO₂ production is completely avoided.

5CO+1H₂+2H₂O→1C₂H₅OH+3CO₂(ΔG°=−204.80 kJ/mol ethanol)

4CO+2H₂+1H₂O→1C₂H₅OH+2CO₂(ΔG°=−184.70 kJ/mol ethanol)

3CO+3H₂→1C₂H₅OH+1CO₂(ΔG°=−164.60 kJ/mol ethanol)

“Stream” refers to any substrate which is capable of being passed, for example, from one process to another, from one module to another, and/or from one process to a carbon capture means.

“Reactants” as used herein refer to a substance that takes part in and undergoes change during a chemical reaction. In particular embodiments, the reactants include but are not limited to CO and/or H2.

“Microbe inhibitors” as used herein refer to one or more constituent that slows down or prevents a particular chemical reaction or another process including the microbe. In particular embodiments, the microbe inhibitors include, but are not limited to, oxygen (O2), hydrogen cyanide (HCN), acetylene (C₂H₂), and BTEX (benzene, toluene, ethylbenzene, xylene).

“Catalyst inhibitor”, “adsorbent inhibitor”, and the like, as used herein, refer to one or more substance that decreases the rate of, or prevents, a chemical reaction. In particular embodiments, the catalyst and/or adsorbent inhibitors may include but are not limited to, hydrogen sulfide (H₂S) and carbonyl sulfide (COS).

“Removal process”, “removal module”, “clean-up module”, and the like includes technologies that are capable of either converting and/or removing microbe inhibitors and/or catalyst inhibitors from the gas stream. In particular embodiments, catalyst inhibitors must be removed by an upstream removal module in order to prevent inhibition of one or more catalyst in a downstream removal module.

The term “constituents”, “contaminants”, and the like, as used herein, refers to the microbe inhibitors, and/or catalyst inhibitors that may be found in the gas stream. In particular embodiments, the constituents include, but are not limited to, sulphur compounds, aromatic compounds, alkynes, alkenes, alkanes, olefins, nitrogen compounds, phosphorous-containing compounds, particulate matter, solids, oxygen, halogenated compounds, silicon-containing compounds, carbonyls, metals, alcohols, esters, ketones, peroxides, aldehydes, ethers, and tars.

The term “treated gas”, “treated stream” and the like refers to the gas stream that has been passed through at least one removal module and has had one or more constituent removed and/or converted.

The term “desired composition” is used to refer to the desired level and types of components in a substance, such as, for example, of a gas stream, including but not limited to syngas. More particularly, a gas is considered to have a “desired composition” if it contains a particular component (e.g. CO, H₂, and/or CO₂) and/or contains a particular component at a particular proportion and/or does not contain a particular component (e.g. a contaminant harmful to the microorganisms) and/or does not contain a particular component at a particular proportion. More than one component may be considered when determining whether a gas stream has a desired composition.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

The term “carbon capture” as used herein refers to the sequestration of carbon compounds including CO₂ and/or CO from a stream comprising CO₂ and/or CO and either:

converting the CO₂ and/or CO into products; or

converting the CO₂ and/or CO into substances suitable for long-term storage; or

trapping the CO₂ and/or CO in substances suitable for long-term storage;

or a combination of these processes.

In certain embodiments, the fermentation is performed in the absence of carbohydrate substrates, such as sugar, starch, lignin, cellulose, or hemicellulose.

The microorganism of the disclosure may be cultured with the gas stream to produce one or more products. For instance, the microorganism of the disclosure may produce or may be engineered to produce ethanol (WO 2007/117157), acetate (WO 2007/117157), 1-butanol (WO 2008/115080, WO 2012/053905, and WO 2017/066498), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2014/036152 and WO 2017/066498), 1-hexanol (WO 2017/066498), 1-octanol (WO 2017/066498), chorismate-derived products (WO 2016/191625), 3-hydroxybutyrate (WO 2017/066498), 1,3-butanediol (WO 2017/066498), 2-hydroxyisobutyrate or 2-hydroxyisobutyric acid (WO 2017/066498), isobutylene (WO 2017/066498), adipic acid (WO 2017/066498), 1,3-hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and monoethylene glycol (WO 2019/126400). In certain embodiments, microbial biomass itself may be considered a product. These products may be further converted to produce at least one component of diesel, jet fuel, and/or gasoline. Additionally, the microbial biomass may be further processed to produce a single cell protein (SCP).

A “single cell protein” (SCP) refers to a microbial biomass that may be used in protein-rich human and/or animal feeds, often replacing conventional sources of protein supplementation such as soymeal or fishmeal. To produce a single cell protein or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients.

An “excipient” may refer to any substance that may be added to the microbial biomass to enhance or alter the form, properties, or nutritional content of the animal feed. For example, the excipient may comprise one or more of a carbohydrate, fiber, fat, protein, vitamin, mineral, water, flavor, sweetener, antioxidant, enzyme, preservative, probiotic, or antibiotic. In some embodiments, the excipient may be hay, straw, silage, grains, oils or fats, or other plant material. The excipient may be any feed ingredient identified in Chiba, Section 18: Diet Formulation and Common Feed Ingredients, Animal Nutrition Handbook, 3rd revision, pages 575-633, 2014.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism but is not produced by a genetically unmodified microorganism from which the genetically modified microorganism is derived.

“Selectivity” refers to the ratio of the production of a target product to the production of all fermentation products produced by a microorganism. The microorganism of the disclosure may be engineered to produce products at a certain selectivity or at a minimum selectivity. In one embodiment, a target product account for at least about 5%, 10%, 15%, 20%, 30%, 50%, or 75% of all fermentation products produced by the microorganism of the disclosure. In one embodiment, the target product accounts for at least 10% of all fermentation products produced by the microorganism of the disclosure, such that the microorganism of the disclosure has a selectivity for the target product of at least 10%. In another embodiment, the target product accounts for at least 30% of all fermentation products produced by the microorganism of the disclosure, such that the microorganism of the disclosure has a selectivity for the target product of at least 30%.

“Increasing the efficiency,” “increased efficiency,” and the like include, but are not limited to, increasing growth rate, product production rate or volume, product volume per volume of substrate consumed, or product selectivity. Efficiency may be measured relative to the performance of parental microorganism from which the microorganism of the disclosure is derived.

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

In certain embodiments, the fermentation is performed in the absence of light or in the presence of an amount of light insufficient to meet the energetic requirements of photosynthetic microorganisms. In certain embodiments, the microorganism of the disclosure is a non-photosynthetic microorganism.

As used herein, the terms “fermentation broth” or “broth” refer to the mixture of components in a bioreactor, which includes cells and nutrient media as well as fermentation products and byproducts. As used herein, a “separator” is a module that is adapted to receive fermentation broth from a bioreactor and pass the broth through a filter to yield a “retentate” and a “permeate.” The filter may be a membrane, e.g. a cross-flow membrane or a hollow fibre membrane. The term “permeate” is used to refer to substantially soluble components of the broth that pass through the separator. The permeate will typically contain soluble fermentation products, byproducts, and nutrients. The retentate will typically contain cells. As used herein, the term “broth bleed” is used to refer to a portion of the fermentation broth that is removed from a bioreactor and not passed to a separator.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably recycled back to the bioreactor. The cell-free permeate remaining after target products have been removed is also preferably returned to the bioreactor. Additional nutrients may be added to the cell-free permeate to replenish the medium before it is returned to the bioreactor.

The primary microorganism may be, for example, selected from the group consisting of Acetobacterium, Alkalibaculum, Blautia, Butyribacterium, Clostridium, Eubacterium, Moorella, Oxobacter, Sporomusa, and Thermoanaerobacter. In particular, the primary microorganism may be derived from a parental bacterium selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui. The primary microorganism may also be selected from the group consisting of Acetitomaculum ruminis, Acetoanaerobium noterae, Acetobacterium bakii, Acetobacterium carbinolicum, Acetobacterium dehalogenans, Acetobacterium fimetarium, Acetobacterium malicum, Acetobacterium paludosum, Acetobacterium tundrae, Acetobacterium wieringae, Acetobacterium woodii, Acetohalobium arabicum, Acetonema longum, Blautia coccoides, Blautia hydrogenotrophica, Blautia producta, Blautia schinkii, Butyribacterium methylotrophicum, Clostridium aceticum, Clostridium autoethanogenum, Clostridium carboxidivorans, Clostridium drakei, Clostridium formicoaceticum, Clostridium glycolicum, Clostridium ljungdahlii, Clostridium magnum, Clostridium mayombei, Clostridium methoxybenzovorans, Clostridium ragsdalei, Clostridium scatologenes, Eubacterium aggregans, Eubacterium limosum, Morellla mulderi, Morella thermoacetica, Morella thermoautotrophica, Oxobacter pfennigii, Sporomusa acidovorans, Sporomusa aerivorans, Sporomusa malonica, Sporomusa ovata, Sporomusa paucivorans, Sporomusa rhizae, Sporomusa silvacetica, Sporomusa spaeroides, Sporomusa termitida, Thermoacetogenium phaeum, Thermoanaerobacter kivui, Acetobacterium, Moorella, Moorella sp HUC22-1, Moorella thermoacetica, Clostridium, Clostridium carboxidivorans, Clostridium drakei, Clostridium acidiurici, Pyrococcus, Pyrococcus furiosus, Eubacterium, Eubacterium limosum, Desulfobacterium, Cabroxydothermus, Acetogenium, Acetoanaerobium, Butyribaceterium, Butyribacterium methylotrophicum, Peptostreptococcus, Ruminococcus, Oxobacter, Oxobacter pfennigii, Methanosarcina, Carboxydothermus, Eubacterium limosum, Desulfotomaculum orientis, Peptococcus glycinophilus, Peptococcus magnets, Ignicoccus hospitalis, Thermoanaerobacter kivui, and Thermoacetogenium phaeum. The microorganism may also be selected from Table 1 of Schiel-Bengelsdorf, FEBS Letters 586: 2191-2198, 2012. In one embodiment, the primary microorganism is Acetobacterium woodii. In another embodiment, the primary microorganism is a Wood-Ljungdahl microorganism. “Wood-Ljungdahl” refers to the Wood-Ljungdahl pathway of carbon fixation as described, e.g., by Ragsdale, Biochim Biophys Acta, 1784: 1873-1898, 2008. “Wood-Ljungdahl microorganisms” refers, predictably, to microorganisms containing the Wood-Ljungdahl pathway. The primary microorganism often contains a native Wood-Ljungdahl pathway.

In another embodiment, the primary microorganism is an acetogen. “Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO2, (2) terminal electron-accepting, energy conserving process, (3) mechanism for the fixation (assimilation) of CO2 in the synthesis of cell carbon (Drake, Acetogenic Prokaryotes, In: The Prokaryotes, 3rd edition, p. 354, New York, N.Y., 2006). All naturally occurring acetogens are C1-fixing, anaerobic, autotrophic, and non-methanotrophic.

The primary microorganism is capable of consuming a substrate (a “primary substrate”) that provides carbon and/or energy. Typically, the primary substrate is gaseous and comprises a C1-carbon source, for example, CO, CO₂, and/or CH₄. Preferably, the primary substrate comprises a C1-carbon source of CO or CO+CO₂. The primary substrate may further comprise other non-carbon components, such as H₂, N₂, or electrons.

In an embodiment, the primary substrate comprises CO₂ and H₂. In an embodiment, the H₂ is renewable H₂. For example, the primary substrate may comprise about 1-80 or 1-30 mol % CO₂. In some embodiments, the primary substrate may comprise less than about 20, 15, 10, or 5 mol % CO₂. The primary substrate may comprise about 1, 2, 5, 10, 15, 20, or 30 mol % H₂. In some embodiments, the primary substrate may comprise a relatively high amount of H₂, such as about 60, 70, 80, or 90 mol % H₂. The primary substrate may also comprise some amount of CO and/or some amount of inert gases, such as N₂.

The primary substrate may be a waste gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the substrate and/or C1-carbon source may be captured from the industrial process before it is emitted into the atmosphere, using any convenient method.

The primary substrate may be also syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The composition of the substrate may have a significant impact on the efficiency and/or cost of the reaction. For example, the presence of oxygen (O₂) may reduce the efficiency of an anaerobic fermentation process, and oftentimes the fermentation will be anaerobic. Depending on the composition of the substrate, it may be desirable to treat, scrub, or filter the substrate to remove any undesired impurities, such as toxins, undesired components, or dust particles, and/or increase the concentration of desirable components.

In certain embodiments, the fermentation is performed in the absence of carbohydrate substrates, such as sugar, starch, lignin, cellulose, or hemicellulose.

The fermentation produces at least one product (a “product”). Typically, this product will be acetate, although the fermentation may also produce additional products such as ethanol and lactate. Microbial biomass may also be considered a product, as it has potential applications in animal feed and fertilizers. Importantly, the terms “acetate” and “acetic acid” may be used interchangeably herein and “lactate” and “lactic acid” may be used interchangeably herein. The product or products then need to be separated and recovered from the fermentation broth.

The term “fermentation” should be interpreted as a metabolic process that produces chemical changes in a substrate. For example, a fermentation process receives one or more substrates and produces one or more products through utilization of one or more microorganisms. The term “fermentation” should be interpreted as the process which receives one or more substrates and produces one or more products through the utilization of one or more microorganisms. Often the fermentation process includes the use of one or more bioreactor. The fermentation process may be described as either “batch” or “continuous”. “Batch fermentation” is used to describe a fermentation process where the bioreactor is filled with raw material, e.g. the carbon source, along with microorganisms, where the products remain in the bioreactor until fermentation is completed. In a “batch” process, after fermentation is completed, the products are extracted, and the bioreactor is cleaned before the next “batch” is started. “Continuous fermentation” is used to describe a fermentation process where the fermentation process is extended for longer periods of time, and product and/or metabolite is extracted during fermentation. Preferably the fermentation process is continuous. Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. In one embodiment the aqueous culture medium is an anaerobic microbial growth medium, such as a minimal anaerobic microbial growth medium. Suitable media are well known in the art.

The culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Separated microbial cells are preferably recycled back to the bioreactor. The cell-free permeate remaining after target products have been removed is also preferably returned to the bioreactor. Additional nutrients may be added to the cell-free permeate to replenish the medium before it is returned to the bioreactor.

In one embodiment, acetate and/or lactate is recovered from the fermentation broth using the separation technique of ion exchange adsorption. The process described in this disclosure has the advantage of being able to separate the conjugate base of the acid from the fermentation broth, without the need to substantially shift the pH of the broth, which could affect the microorganisms. In this way acetate and/or lactate or another conjugate base of a low molecular weight organic acid can be separated from live broth and the live broth containing the live microorganisms may be returned to the bioreactor. Specifically, the separation is accomplished by integrated expended bed adsorption and simulated moving bed technology.

Adsorption technology separates by interacting a mobile liquid stream containing one or more target compounds with a stationary phase. One adsorption mechanism is ion exchange which utilizes an ion exchange resin. The ion exchange operation may be operated continuously. In expanded bed technologies, the bed of resin is fluidized by upward feed flow and the resulting bed voids allow particulate biomass to flow through the bed without clogging the bed and remaining trapped in the bed of ion exchange resin. Thus unclarified, or raw, live broth may be passed through the expanded bed without the biomass becoming physically trapped. The resin selection is also important as some microorganisms have an affinity to specific resins. Therefore, the resin may be selected to ion exchange with the targeted molecule for separation and must also be compatible with the specific microorganism of the fermentation broth. Finally, in one embodiment, the expanded bed is operated in a simulated moving bed mode (SMB). SMB is well known and is used to obtain optimum resin utilization and superior resolution. In SMB, the resin flow rate is simulated by periodically shifting different inlet and outlet ports in the direction of the fluid flow. Expanded bed adsorption integrated with simulated moving bed technology is described in Chem. Eng. Technol. 2018, 41, No. 12, 2393-2401.

The clarified or unclarified fermentation broth of this disclosure is passed, in part or in whole, through a separation unit containing an ion exchange resin and operated as an expanded adsorption bed in the simulated moving bed mode. The ion exchange resin is selected to ion exchange with acetate or lactate or both in the fermentation broth. The ion exchange resin is further selected to be compatible with the microorganism of the fermentation broth. Compatibility is based, at least in part, on the nature of the microorganism and its natural tendency to stick or adhere to the resin. Ion exchange resins where the microorganism does not stick or adhere to the resin are desired. Resins may be any suitable type include the gel-type or the porous resins such as macroporous polystyrene resins. The ion exchange resins may be of the strong anion exchange resin type. Suitable examples of a class of strong anion exchange resins are those in the chloride form sold under the trade names of AG 1-X8 and AG 1-X2 available from Bio Rad, Amberlite HPR9200 C1 available from Dow, Amberlite IRA900 C1 available from DuPont, and Diaion PA408 C1 available from Mitsubishi Chemical.

A specific example includes the separation of acetate from a fermentation broth resulting from the culturing of a microorganism of the genus Clostridium in the presence of a gas substrate the separation using an ion exchange resin in an expanded adsorption bed operated in the simulated moving bed mode. Another specific example includes the separation of lactate resulting from the culturing of a microorganism of the genus Clostridium in the presence of a gas substrate, the separation using an ion exchange resin in an expanded adsorption bed operated in the simulated moving bed mode. Another specific example includes the separation of both acetate and lactate from a fermentation broth resulting from the culturing of a microorganism of the genus Clostridium in the presence of a gas substrate the separation using an ion exchange resin in an expanded adsorption bed operated in the simulated moving bed mode. The acetate, lactate, or both maybe recovered, and the ion exchange resin regenerated as is customary for the ion exchange resin selected. For example, the acetate, lactate, or both may be recovered using solutions containing nitrates or chlorides.

Because the fermentation broth is aqueous, acetic acid and lactic acid are only partially dissociated. Therefore, after the acetate and lactate are removed by ion exchange, remaining acetic acid and or lactic acid recirculates with the fermentation broth and may further dissociate to provide additional acetate and lactate that may be separated and recovered in additional passes through the ion exchange separation step. Depending upon the specifics of the application, pH adjustment may be required.

With the acetate from the fermentation broth separated and recovered, the acetate may be converted to acetic acid. The acetic acid may be catalytically reacted with ethylene and oxygen to form vinyl acetate. The catalyst may be a palladium catalyst.

2C2H2+2CH3CO2H+O2→2CH3CO2CHCH2+2H2O

In one embodiment, the ethylene used in the reaction to form vinyl acetate may be at least partially derived from ethanol that is the result of gas fermentation of a carbon oxide containing gas. The carbon oxide containing gas may be as described above.

Vinyl acetate is also known as vinyl acetate monomer (VAM) and may be polymerized to give polyvinyl acetate (PVA) or polymerized and reacted to form polyvinyl alcohol. The vinyl acetate may also be polymerized with other monomers to produce various copolymers including ethylene-vinyl acetate, vinyl acetate-acrylic acid, polyvinyl chloride acetate, and polyvinylpyrrolidone. The vinyl acetate may also be reacted with bromine to form dibromide, reacted with hydrogen halides to form 1-haloethyl acetates, reacted with acetic acid in the presence of platinum catalysts to give ethylidene diacetate. The vinyl acetate may undergo transesterification to give vinyl ethers.

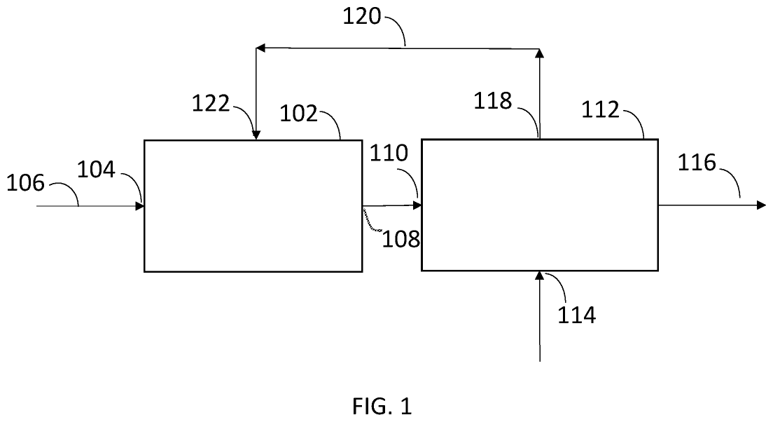

A biological conversion apparatus is also disclosed herein and comprises a bioreactor system and the separation zone described above. Turning to FIG. 1, inlet 104 conducts a gas substrate in feed line 106 to bioreactor 102. Bioreactor 102 contains culture medium and microorganisms to metabolize a carbon source in the substrate and produce a product. Outlet 108 allows the fermentation broth from the bioreactor, which includes the culture medium, the microorganisms, the product(s), to pass from the bioreactor. Inlet 110 of separation zone 112 is in fluid communication with outlet 108. Separation zone 112 contains an expanded bed of ion exchange resin in a simulated moving bed configuration. In separation zone 112 the product(s) are ion exchanged with the resin and thereby retained in the bed. The remaining portion of the fermentation broth including the microorganisms pass through the ion exchange resin to outlet 118 and may be recycled to bioreactor 102 through line 120 to recycle inlet 122 of bioreactor 102. Separation zone has inlet 114 which is in fluid communication with a regenerate source in order to regenerate the ion exchange resin and release the product. Separation zone 112 also has product outlet 116 for the recovery of the released product. Optionally, the counter ion released from the ion exchange resin during the ion exchange process may be removed from the fermentation broth prior to recycling to the bioreactor. Details of simulated moving bed operation is known and not discussed here.

In another embodiment, the biological conversion apparatus comprises a bioreactor system and the separation zone described above, as well as a microbial biomass separation zone positioned between the bioreactor system and the separation zone. In this embodiment, only a portion of the fermentation broth of the bioreactor is passed to the separation zone containing the ion exchange resin. The microbial biomass separation zone may employ a technique such as membrane separation to separate a microbial biomass containing portion of the fermentation broth when may then be recycled to the bioreactor. The remainder of the fermentation broth may then be passed to the separation zone containing the ion exchange resin. The separation zone containing the ion exchange resin may be operated in a variety of different modes of operation such as in a fixed bed mode, in a swing bed mode using two or more fixed beds, in a simulated moving bed mode, in a moving bed mode, or other modes.

One advantage of removing the microbial biomass before passing the fermentation broth to the separation zone containing the ion exchange resin is that the size of the separation zone may be reduced due to the volume of material processed through the separation zone being reduced. Another advantage is to readily and easily control the exposure of the microbial biomass to the counter ion released from the ion exchange resin during the ion exchange process.

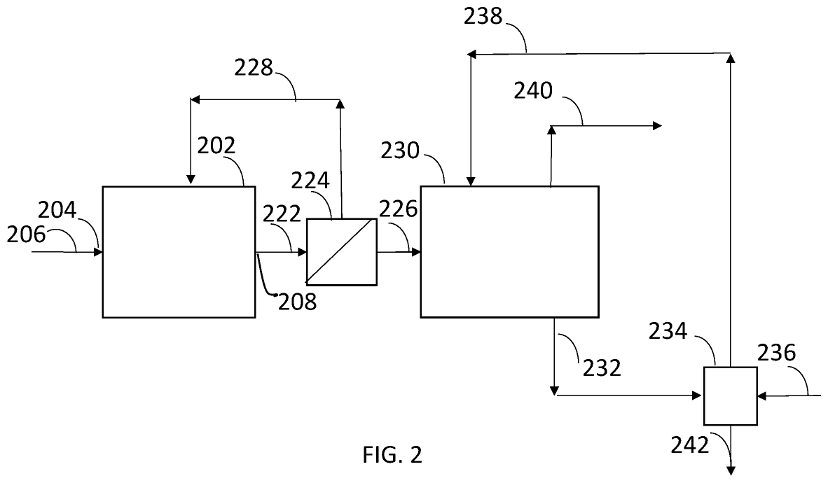

Turning to FIG. 2, inlet 204 conducts a gas substrate in feed line 206 to bioreactor 202. Bioreactor 202 contains culture medium and microorganisms to metabolize a carbon source in the substrate and produce a product. Outlet 208 allows the fermentation broth from the bioreactor, which includes the culture medium, the microorganisms, the product(s), to pass from bioreactor 202 through conduit 222 to microbial biomass separation zone 224. Microbial biomass separation zone 224 is shown in FIG. 2 as a membrane separation zone where the retentate portion of the fermentation broth contains the microbial biomass and the permeate portion of the fermentation broth contains the target component such as acetate or lactate to be separated. The retentate portion of the fermentation broth containing the microbial biomass is recycled in line 228 to bioreactor 202. Permeate portion of the fermentation broth containing product(s) to be separated, such as acetate, is conducted from microbial biomass separation zone 224 to separation zone 230 via line 226. Other microbial biomass separation techniques may be employed, and the membrane separation technique shown is merely exemplary.

Separation zone 230 contains at least one fixed bed of ion exchange resin. It may be advantageous for separation zone 230 to contain at least two fixed beds of ion exchange resin so that one fixed bed may be on-line and in operation while the other is being regenerated. The product(s) are ion exchanged with the resin and thereby retained in the bed. The portion of the fermentation broth conducted in line 226 enters separation zone 230 and contacts the ion exchange resin where the product(s) are ion exchanged with the resin and thereby retained in the bed. The non-retained portion of the fermentation broth, which now additionally contains the counter ion from the resin, passes through the ion exchange resin bed and is removed from separation zone 230 in effluent stream 232 and is routed to treatment unit 234 for treatment to be used as regenerant for the ion exchange resin. Treatment may include reacting away a specific counter ion in favor of another counter ion by addition of solution via line 236 and removal of the reaction product containing the less favored counter ion in line 242. Regenerate is passed to separation zone 230 in line 238 to contact the ion exchange resin and regenerate the ion exchange resin and release the desired product. The now separated desired product is removed from separation zone 230 in product recovery line 240.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Different embodiments of this disclosure are described herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method for separating a target component from a fermentation broth comprising:
a. fermenting a gas substrate and a microorganism to generate a fermentation broth comprising the microorganism and the target component;
b. passing the fermentation broth to a separation unit having an ion exchange resin in a continuous ion exchange simulated moving bed;
c. selectively retaining the target component through ion exchange with the resin and passing the microorganism through the continuous ion exchange simulated moving bed; and
d. regenerating the ion exchange resin and recovering the target component.

2. The method of claim 1 wherein the target component is a conjugate base of a low molecular weight organic acid.

3. The method of claim 1 wherein the target component is acetate, lactate, or both.

4. The method of claim 1 wherein the continuous ion exchange simulated moving bed is an expanded bed.

5. The method of claim 1 wherein the ion exchange resin is a strong anion exchange resin.

6. The method of claim 1 wherein the microorganism is derived from a parental microorganism selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui.

7. The method of claim 1 wherein the microorganism is a member of the genus Clostridium.

8. The method of claim 1 wherein the microorganism is derived from Clostridium autoethanogenum, Clostridium ljungdahlii, Clostridium ragsdalei, or Clostridium coskatii.

9. The method of claim 1 wherein the gas substrate is industrial waste gas, industrial off gas, synthesis gas derived from gasified waste, synthesis gas derived from gasified biomass, or any combination thereof.

10. The method of claim 1 wherein the target component is reacted to form one or more products.

11. The method of claim 10 wherein the target compound is acetate and the one or more products is vinyl acetate.

12. The method of claim 11 further comprising reacting the vinyl acetate to form polyvinyl acetate or polyvinyl alcohol.

13. The method of claim 12 further comprising reacting the polyvinyl acetate or polyvinyl alcohol to form a polymer, a copolymer, an adhesive, a coating, a paint, a film, a textile, a foam, a wire insulation or a cable insulation.

14. A method for separating a target component from a fermentation broth comprising:
a. fermenting a gas substrate and a microorganism to generate a fermentation broth comprising the microorganism and the target component;
b. passing the fermentation broth to a first separation zone to separate and recycle a first portion of the fermentation broth comprising the microorganism to the bioreactor;
c. passing a second portion of the fermentation broth to a second separation zone comprising ion exchange resin;
d. selectively retaining the target component through ion exchange with the resin and passing remainder through the second separation zone; and
e. regenerating the ion exchange resin with a regenerate and recovering the target component.

15. The method of claim 14 wherein the regenerate comprises at least a portion of the remainder or is derived from the remainder.

16. The method of claim 14 wherein the target component is a conjugate base of a low molecular weight organic acid.

17. The method of claim 14 wherein the target component is acetate, lactate, or both.

18. The method of claim 14 wherein the ion exchange resin is a strong anion exchange resin.

19. The method of claim 14 wherein the gas substrate is industrial waste gas, industrial off gas, synthesis gas derived from gasified waste, synthesis gas derived from gasified biomass, or any combination thereof.

20. The method of claim 14 wherein the target component is reacted to form one or more products.

21. The method of claim 20 wherein the target compound is acetate and the one or more products is vinyl acetate.

22. The method of claim 21 further comprising reacting the vinyl acetate to form polyvinyl acetate or polyvinyl alcohol.

23. The method of claim 22 further comprising reacting the polyvinyl acetate or polyvinyl alcohol to form a polymer, a copolymer, an adhesive, a coating, a paint, a film, a textile, a foam, a wire insulation or a cable insulation.

24. A biological conversion apparatus comprising:
a. a bioreactor system comprising an inlet to a bioreactor containing a culture medium and microorganisms to metabolize a carbon source in a substrate and produce a product and an outlet from the bioreactor; and
b. a separation zone comprising a first inlet in fluid communication with the outlet of the bioreactor, a bed of ion exchange resin in a simulated moving bed configuration, a second inlet in fluid communication with a regenerate source, an outlet in fluid communication with the bioreactor system, and a product outlet.

25. The biological conversion apparatus of claim 24 wherein the bed of ion exchange resin is an expanded bed of ion exchange resin.

26. A biological conversion apparatus comprising:
a. a bioreactor system comprising an inlet to a bioreactor containing a culture medium and microorganisms to metabolize a carbon source in a substrate and produce a product and an outlet from the bioreactor;
b. a first separation zone comprising an inlet in fluid communication with the outlet of the bioreactor, a membrane for the separation of microbial biomass, a retentate outlet in fluid communication with the bioreactor, and a permeate outlet; and
c. a second separation zone comprising a first inlet in fluid communication with the permeate outlet of the first separation zone, at least one bed of ion exchange resin, a second inlet in fluid communication with a regenerate source, an outlet in fluid communication with the regenerate source, and a product outlet.
